(19) United States
(12) Patent Application Publication
(10) Pub. No.: US 2018/0263532 A1
(43) Pub. Date: Sep. 20, 2018

(54) TECHNOLOGIES FOR INDICATING DETECTION OF TOE WALKING

(71) Applicants: Samantha Smulyan, Indianapoliks, IN (US); Heather Smulyan, Indianapolis, IN (US); Jeff Smulyan, Indianapolis, IN (US)

(72) Inventors: Samantha Smulyan, Indianapoliks, IN (US); Heather Smulyan, Indianapolis, IN (US); Jeff Smulyan, Indianapolis, IN (US)

(21) Appl. No.: 15/464,105

(22) Filed: Mar. 20, 2017

(51) Int. Cl. A61B 5/11; A43B 3/00; A43B 17/00; A61B 5/00

(52) U.S. Cl. CPC A61B 5/112; A43B 3/0005; A43B 17/00; A61B 2562/0219; A61B 5/7271; A61B 5/6802; A61B 2562/0252; A61B 5/0015

(57) ABSTRACT

Technologies for indicating the detection of toe walking include a gait monitoring device that includes one or more sensors usable to collect gait data relative to the gait monitoring device and vibration circuitry that includes a vibrating motor usable to provide haptic feedback in the form of a vibration. The gait monitoring device is configured to analyze the gait data collected from the one or more sensors, detect a toe walking event as a function of the analysis of the collected gait data, and enable, subsequent to having detected the toe walking event, the vibrating motor for a predetermined period of time. Additional embodiments are described herein.

### BACKGROUND OF THE DISCLOSED EMBODIMENTS

A human walking cycle, or bipedal gait cycle, describes the sequence of events exhibited by the lower-limbs (i.e., legs and feet) during normal walking. The bipedal gait cycle is comprised of alternating stance phases: a stance phase (i.e., when all or part of at least one foot is in contact with the support surface) and a swing phase (i.e., when at least one foot is not in contact with the support surface). The stance phase generally constitutes roughly 60% of the gait cycle and is typically divided into five or more period which can include: an initial contact period commonly referred to as a heel strike; a loading response period (i.e., the foot being flat with the support surface); a mid-stance period; a terminal stance period (i.e., when the heel leaves the support surface); and a toe off period, or pre-swing period. The swing phase constitutes the remainder of the gait cycle at roughly 40% and is typically divided into three periods: an initial swing period; a mid swing period, and a terminal swing period.

However, not all humans exhibit such a normal gait cycle. While some humans can attribute their abnormal gait cycle to a particular medical diseases (e.g., a neuromuscular disease), others do it unknowingly (e.g., out of habit). Prolonged exposure to abnormal gait cycles may have undesirable consequences, such as the shortening of tendons, muscular atrophy, etc. For example, some humans strike with their heel first, a condition commonly referred to as toe walking. Toe walking is generally considered a gait abnormality in which the forefoot is primarily engaged with the support surface throughout the gait cycle, including the heel strike period of the stance phase. While most commonly exhibited by children, such a condition may result from habit (e.g., idiopathic toe walking) or a medical condition. For idiopathic toe walkers, toe walking is the manifestation of the toe walking gait pattern with no known underlying pathological foundation. As such, toe walking may be a correctable condition if the toe walker is able to recognize when they are toe walking.

Accordingly, there exists a need for improvements in technologies for indicating detection of toe walking.

### SUMMARY OF THE DISCLOSED EMBODIMENTS

In one aspect, a method for indicating detection of toe walking includes receiving, by a gait monitoring device, gait data from one or more sensors of the gait monitoring device; detecting, by the gait monitoring device, a toe walking event as a function of the received gait data; and enabling, by the gait monitoring device and subsequent to detecting the toe walking event, vibration circuitry of the gait monitoring device for a predetermined period of time.

In some embodiments, the method further includes determining, by the gait monitoring device, whether the gait monitoring device is being moved in a bipedal locomotion, wherein detecting the toe walking event is subsequent to having determined the gait monitoring device is being moved in the bipedal locomotion. In other embodiments, determining whether the gait monitoring device is being moved in the bipedal locomotion comprises comparing at least a portion of the gait data received from an inertial measurement unit sensor of the gait monitoring device and one or more movement threshold values. In still other embodiments, the one or more movement threshold values include at least one of a minimum acceleration threshold, an orientation threshold, and a magnetic flux threshold.

In some embodiments, the method further includes determining, by the gait monitoring device, a present phase and a present period of a gait cycle of a user of the gait monitoring device, wherein detecting the toe walking event is subsequent to having determined the present phase of the gait cycle corresponds to a stance phase and the present period of the gait cycle corresponds to an initial contact period. In other embodiments, determining the present phase and the present period of the gait cycle of the user comprises comparing at least a portion of the gait data received from a load cell sensor of the gait monitoring device and one or more load threshold values. In still other embodiments, the one or more load threshold values include at least one of a minimum detected weight, a minimum detected force, a minimum electrical charge.

In some embodiments, the method further includes transmitting, by the gait monitoring device, an indication to a toe walking tracker application presently executing on a mobile computing device that is wirelessly coupled to the gait monitoring device. In other embodiments, detecting the toe walking event as a function of the received gait data comprises comparing at least a portion of the gait data received from a load cell sensor of the gait monitoring device and one or more load threshold values.

In another aspect, a gait monitoring device for indicating detection of toe walking includes one or more sensors usable to collect gait data relative to the gait monitoring device; vibration circuitry that includes a vibrating motor usable to provide haptic feedback in the form of a vibration; one or more computer-readable medium comprising instructions; one or more processors coupled with the one or more computer-readable medium. Additionally, the one or more processors are configured to execute the instructions to analyze the gait data collected from the one or more sensors; detect a toe walking event as a function of the analysis of the collected gait data; and enable, subsequent to having detected the toe walking event, the vibrating motor for a predetermined period of time.

In some embodiments, the one or more processors are further configured to execute the instructions to determine whether the gait monitoring device is being moved in a bipedal locomotion, wherein to detect the toe walking event is subsequent to having determined the gait monitoring device is being moved in the bipedal locomotion. In other embodiments, to determine whether the gait monitoring device is being moved in the bipedal locomotion comprises to compare at least a portion of the gait data received from an inertial measurement unit sensor of the gait monitoring device and one or more movement threshold values. In still other embodiments, the one or more movement threshold values include at least one of a minimum acceleration threshold, an orientation threshold, and a magnetic flux threshold.

In some embodiments, the one or more processors are further configured to execute the instructions to determine a present phase and a present period of a gait cycle of a user of the gait monitoring device, wherein to detect the toe walking event is subsequent to having determined the present phase of the gait cycle corresponds to a stance phase and the present period of the gait cycle corresponds to an initial contact period. In other embodiments, the one or more sensors includes one or more load cell sensors, and wherein to determine the present phase and the present period of the gait cycle of the user comprises comparing at least a portion of the gait data received from at least one of the load cell sensors and one or more load threshold values. In still other embodiments, the one or more load threshold values include at least one of a minimum detected weight, a minimum detected force, a minimum electrical charge.

In some embodiments, the one or more processors are further configured to transmit an indication to a toe walking tracker application presently executing on a mobile computing device that is wirelessly coupled to the gait monitoring device. In other embodiments, the one or more sensors includes one or more load cell sensors, and wherein to detect the toe walking event as a function of the received gait data comprises comparing at least a portion of the gait data received from at least one of the load cell sensors and one or more load threshold values.

### BRIEF DESCRIPTION OF DRAWINGS

The embodiments and other features, advantages and disclosures contained herein, and the manner of attaining them, will become apparent and the present disclosure will be better understood by reference to the following description of various exemplary embodiments of the present disclosure taken in conjunction with the accompanying drawings, wherein:

FIG. 1 is a simplified illustration of at least one embodiment of an overhead view a system for indicating detection of toe walking in which a gait monitoring device is embedded in an insole;

FIG. 2 is a simplified illustration of at least one embodiment of a profile view the system of FIG. 1 in which the insole is placed into a shoe;

FIG. 3 is a simplified block diagram of at least one embodiment of a system for tracking toe walking indications that includes the gait monitoring device of FIGS. 1 and 2 wirelessly communicatively coupled to a mobile computing device;

FIG. 4 is a simplified block diagram of at least one embodiment of the gait monitoring device of FIG. 3;

FIG. 5 is a simplified block diagram of at least one embodiment of the mobile computing device of FIG. 3; and

FIGS. 6A and 6B are a simplified flow diagram of at least one embodiment of a method for indicating detection of toe walking that may be executed by the gait monitoring device of FIGS. 1-4.

### DETAILED DESCRIPTION OF THE DISCLOSED EMBODIMENTS

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

This detailed description is presented in terms of programs, data structures, and/or procedures executed on a single computer or a network of computers. The software programs implemented by the system may be written in any programming language—interpreted, compiled, or otherwise. These languages may include, but are not limited to, VBA, LISP, embedded C, PHP, ASP.net, HTML, HTML5, Ruby, Perl, Java, Python, C++, C#, JavaScript, and/or the Go programming language. It should be appreciated, of course, that one of skill in the art will appreciate that other languages may be used instead, or in combination with the foregoing and that web and/or mobile application frameworks may also be used, such as, for example, Ruby on Rails, Node.js, Zend, Symfony, Revel, Django, Struts, Spring, Play, Jo, Twitter Bootstrap, and others. It should further be appreciated that the systems and methods disclosed herein may be delivered in a software-as-a-service (SaaS) model, made available over a computer network, such as, for example, the Internet. Further, the present disclosure may enable web services, application programming interfaces, and/or service-oriented architecture through one or more application programming interfaces (APIs) or other technologies.

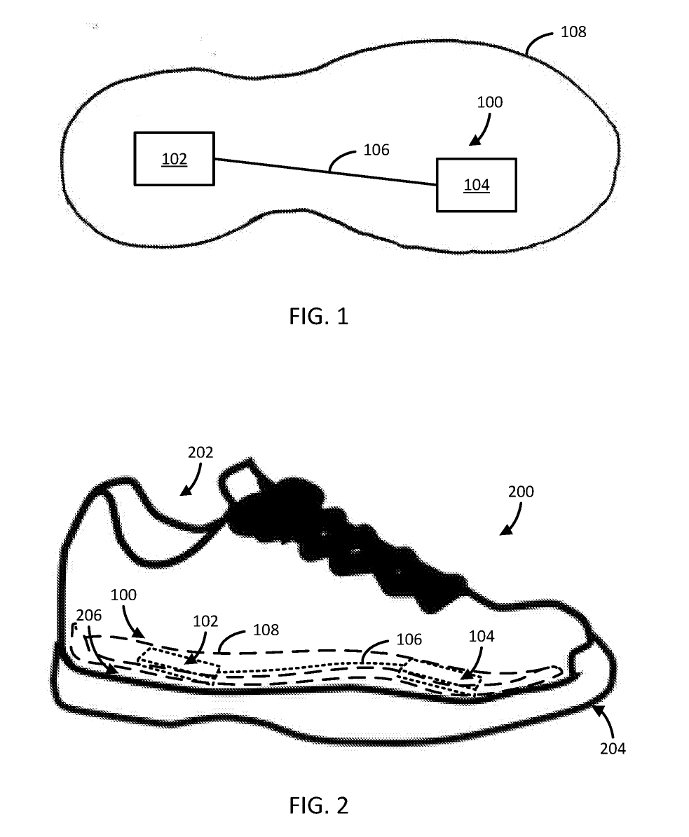

FIG. 1 is an illustrative system for indicating detection of toe walking that includes a gait monitoring device 100. The gait monitoring device 100 includes a heel portion 102 and a forefoot portion 104 communicatively coupled via one or more wires 106 (e.g., a communication bus). In the illustrative embodiment of FIG. 1, the gait monitoring device 100 is embedded into an insole 108. While the heel portion 102 and the forefoot portion 104 are illustratively shown sized and placed relative to the insole, it should be appreciated that the size and/or placement of one or both of the heel portion 102 and the forefoot portion 104 may be different in other embodiments. Additionally or alternatively, in other embodiments, the heel portion 102 and/or the forefoot portion 104 may be comprised of more than one heel portion 102 and/or forefoot portion 104, respectively.

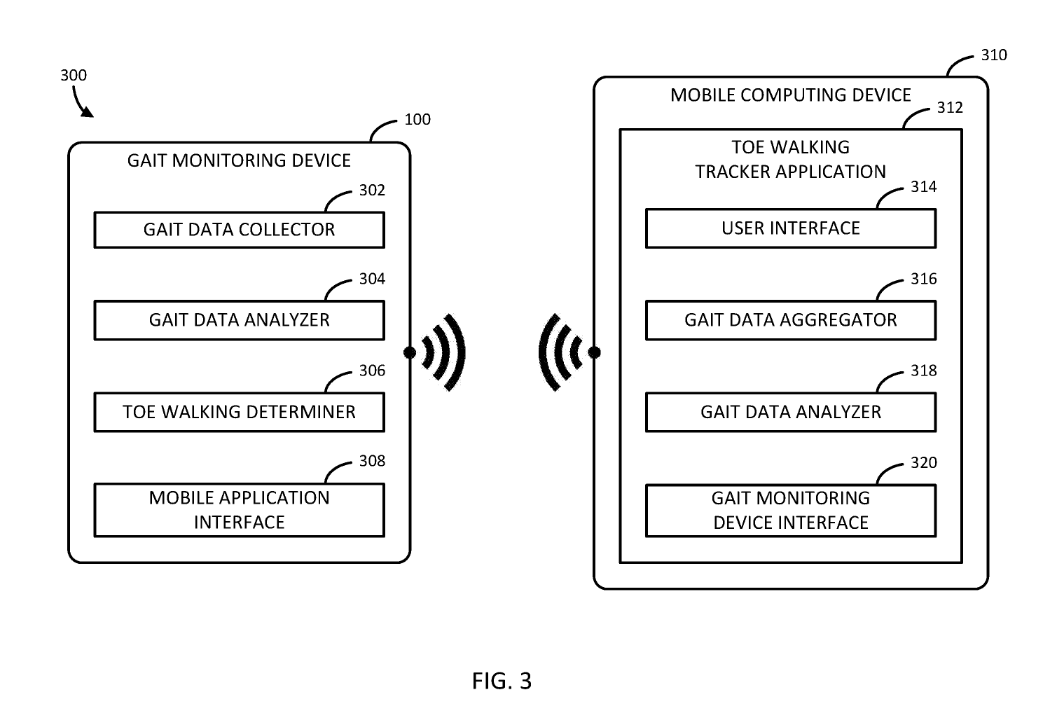

As shown in FIG. 2, the insole 108 of FIG. 1 is illustratively shown as inserted into a shoe 200 (e.g., via the opening 202 of the shoe 200) such that the insole 108 is in contact with a user facing side 206 of a sole 204 of the shoe 200. In use, as will be described in further detail below, the gait monitoring device 100 detects whether a wearer of the shoe 200 is walking on their toes (i.e., toe walking) using gait data collected via one or more sensors of the gait monitoring device 100, including one or more load cell sensors, inertial movement sensors, etc. In other words, the gait monitoring device 100 determines whether a user's heel is in contact with the sole of the shoe at the point of initial contact of the stance phase (i.e., the point at which the shoe initially makes contact with the support surface), as opposed to the user's heel being elevated and the user's forefoot being in contact with the forefoot portion of the shoe at the point of initial contact of the stance phase (i.e., a toe walking event).

It should be appreciated that a toe walking gait pattern (e.g., an equinus or plantarflexed gait pattern) may be attributable to a diagnosed medical disease or disorder (e.g., cerebral palsy, muscular dystrophy or another generalized disease of nerve and muscle). Alternatively, toe walking may be a condition that is habitual (i.e., done out of habit), which may be unnoticed by the toe walker. Such a habitual toe walking condition is commonly referred to as idiopathic toe walking and may be attributable to an underlying neurologic cause.

If the gait monitoring device 100 detects that the wearer is walking on their toes, the gait monitoring device 100 is configured to notify the wearer. To do so, the gait monitoring device 100 is configured to vibrate at least a portion of the gait monitoring device 100 such as to notify the wearer that they are presently toe walking. Accordingly, upon feeling the vibration on their foot, the wearer may consciously adjust their gait pattern to a normal gait cycle (e.g., a heel strike rather than a toe strike at the initial contact period of the stance phase).

In some embodiments, the gait monitoring device 100 may be additionally configured to transmit a notification (i.e., a toe walking notification) to a mobile computing device (see, e.g., the mobile computing device 310 of FIG. 3) with an application installed thereon that is usable to track information associated with the toe walking event. The toe walking notification may include any information related to the toe walking event, such as a time at which the toe walking event was detected. The application may then be used by the toe walker or a monitoring agent thereof (e.g., a medical professional, a parent, etc.). Accordingly, the user of the application may then monitor the times and/or regularity of the detected toe walking events.

As such, the user of the application may diagnose the wearer of the gait monitoring device 100 as a toe walker. As described previously, toe walking may be attributable to a medical condition, in which case the user of the application may monitor the progress of a therapy presently being undergone by the wearer of the gait monitoring device 100, such as may be intended to reduce or altogether eliminate the toe walking by the wearer. As also described previously, toe walking may be idiopathic (i.e., idiopathic toe walking), in which case the user of the application may monitor the effectiveness of the gait monitoring device 100 in reducing or altogether eliminating the toe walking by the wearer

While the gait monitoring device 100 is illustratively shown in one insole 108, it should be appreciated that one gait monitoring device 100 may be embedded in one insole 108 corresponding to the left foot of the wearer, while another gait monitoring device 100 may be embedded in another insole 108 corresponding to the right foot of the wearer. Accordingly, in such embodiments, it should be appreciated each of the gait monitoring devices 100 may be communicatively coupled to the other of the gait monitoring devices 100 such that data and analysis thereof may be passed therebetween.

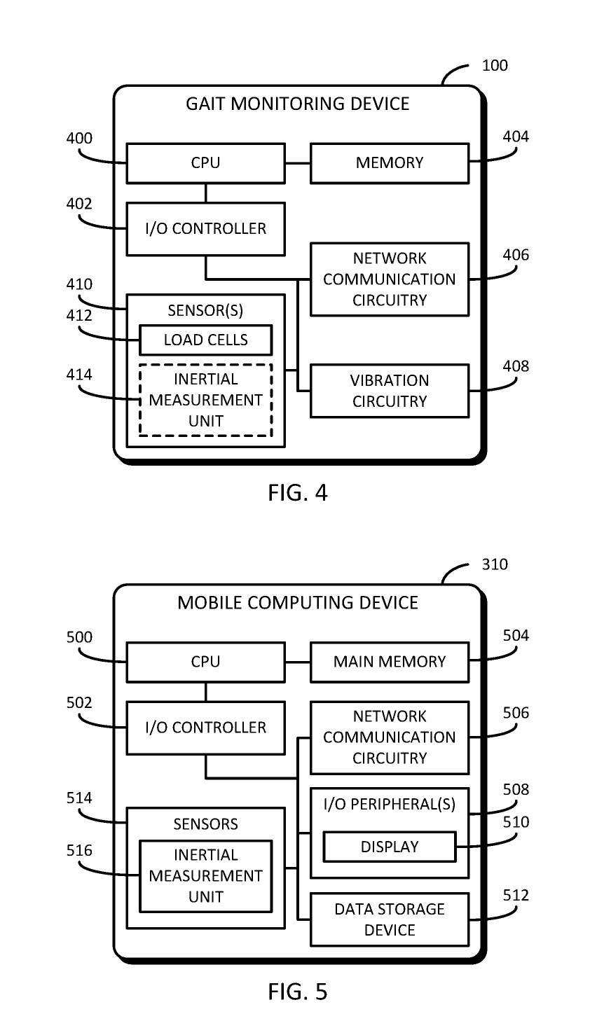

Referring now to FIG. 3, an illustrative system 300 for tracking toe walking indications includes the gait monitoring device 100 of FIG. 1 and a mobile computing device 310. The gait monitoring device 100 may include any type of firmware, hardware, software, circuitry, or combination thereof capable of performing the functions described herein. As shown in FIG. 4, the illustrative gait monitoring device 100 includes a central processing unit (CPU) 400, an input/output (I/O) controller 402, memory 404, network communication circuitry 406, vibration circuitry 408, and a number of sensors 410. It should be appreciated that alternative embodiments may include additional, fewer, and/or alternative components to those shown in the illustrative gait monitoring device 100, such as a power source. It should be further appreciated that one or more of the illustrative components may be combined on a single system-on-a-chip (SoC) on a single integrated circuit (IC).

The CPU 400, or processor, may be embodied as any combination of hardware and circuitry capable of processing data. In some embodiments, the gait monitoring device 100 may include more than one CPU 400. Depending on the embodiment, the CPU 400 may include one processing core (not shown), such as in a single-core processor architecture, or multiple processing cores, such as in a multi-core processor architecture. Irrespective of the number of processing cores and CPUs 400, the CPU 400 is capable of reading and executing program instructions. In some embodiments, the CPU 400 may include cache memory (not shown) that may be integrated directly with the CPU 400 or placed on a separate chip with a separate interconnect to the CPU 400. It should be appreciated that, in some embodiments, pipeline logic may be used to perform software and/or hardware operations (e.g., network traffic processing operations), rather than commands issued to/from the CPU 400.

The I/O controller 402, or I/O interface, may be embodied as any type of computer hardware or combination of circuitry capable of interfacing between input/output devices and the gait monitoring device 100. Illustratively, the I/O controller 402 is configured to receive input/output requests from the CPU 400, and send control signals to the respective input/output devices, thereby managing the data flow to/from the gait monitoring device 100.

The memory 404 may be embodied as any type of computer hardware or combination of circuitry capable of holding data and instructions for processing, such as an internal data storage circuit for a random access memory (RAM) chip (e.g., static RAM (SRAM) or dynamic RAM (DRAM)), non-volatile read-only memory (ROM) (e.g., electrically erasable programmable read-only memory (EEPROM), serial flash, etc.). It should be appreciated that, in some embodiments, one or more components of the gait monitoring device 100 may have direct access to at least a portion of the memory 404, such that certain data may be stored via direct memory access (DMA) independently of the CPU 400.

The network communication circuitry 406 may be embodied as any type of computer hardware or combination of circuitry capable of managing network interfacing communications (e.g., messages, datagrams, packets, etc.) via wireless communication modes. In some embodiments, the network communication circuitry 406 may include low power close-range wireless communication circuitry, such as a Bluetooth® Low Energy (BLE) chipset. Additionally or alternatively, the network communication circuitry 406 may include an embedded Wi-Fi® chip. Accordingly, irrespective of the embodiment, the network communication circuitry 406 may form a portion of self-contained SoC capable of being configured to connect to a mobile computing device (e.g., the mobile computing device 310) or other computing devices, depending on the embodiment.

The vibration circuitry 408 may be embodied as any type of computer hardware or combination of circuitry capable of providing haptic feedback in the form of a vibration, such as a vibration motor (e.g., an eccentric rotating mass (ERM) motor, a pager motor, etc.). In an illustrative example, the vibration circuitry 408 may be embodied as a coin, disc, or pancake vibration motor that may be used due to its smaller form factor and enclosed vibration mechanism. To effectively notify a wearer of a detected toe-walking event, the vibration circuitry 408 may be integrated into the forefront portion 104 of the gait monitoring device 100.

The illustrative sensors 410 include a load cell sensor 412 and, in some embodiments, an inertial measurement unit (IMU) sensor 414. It should be appreciated that, in other embodiments, the sensors 410 may include one or more additional types of sensors. It should be further appreciated that, in some embodiments, additional and/or alternative sensors may be used to measure or assist in measuring one or more of the data points described herein.

The load cell sensor 412 may be embodied as any type of force sensor capable of determining a load as a function of force applied to the load cell sensor 412. In an illustrative example, the load cell sensor 412 may use a bending beam configuration that converts the deformation of one or more thin film strain gauges into an applied force signal. In another illustrative example, the load cell sensor 412 may use one or more piezoresistive force sensors usable to measure force directly as a function of a level of compression between two layers of flexible, printed, piezoresistive ink, and convert the measured force into an electrical charge. It should be appreciated that the type of load cell sensor 412 is not limited to the illustrative examples provided herein, and may additionally and/or alternatively include any other type of force collection sensor technologies.

In an illustrative embodiment, one or more load cell sensor(s) 412 are located in each of the heel portion 102 and the forefoot portion 104 of the gait monitoring device 100. Accordingly, the weight of the monitored wearer's forefoot can be measured when the forefoot is in contact with the one or more load cell sensor(s) 412 of the forefoot portion 104 (e.g., during the stance phase) and the weight of the monitored wearer's heel can be measured when the heel is expected to be in contact with the one or more load cell sensor(s) 412 of the forefoot portion 104 (e.g., during the initial contact period of the stance phase).

In such embodiments in which the IMU sensor 414 is included, the IMU sensor 414 may include one or more software or hardware gyroscopes to measure the orientation of the gait monitoring device 100 (e.g., a 3-axis gyroscope), software or hardware accelerometers to measure proper acceleration of the gait monitoring device 100 (e.g., a 3-axis accelerometer), software or hardware magnetometers to measure the direction of the Earth's magnetic field relative to the gait monitoring device 100 (e.g., a 3-axis magnetometer), or any other type of inertial motion measurement software/hardware usable to perform the functions described herein (e.g., measure motion along three perpendicular linear axes and/or the rotation around each of the three perpendicular linear axes). It should be appreciated that one or more IMU sensor(s) 414 may be located in each of the heel portion 102 and the forefoot portion 104 of the gait monitoring device 100, depending on the embodiment.

The illustrative gait monitoring device 100 includes a gait data collector 302, a gait data analyzer 304, a toe walking determiner 306, and a mobile application interface 308, each of which may be embodied as any type of firmware, hardware, software, circuitry, or combination thereof that is configured to perform the functions described herein. In some embodiments, the gait data collector 302, the gait data analyzer 304, the toe walking determiner 306, and/or the mobile application interface 308 may include one or more computer-readable medium (e.g., the memory 404 and/or any other media storage device) having instructions stored thereon and one or more processors (e.g., the CPU 400) coupled with the one or more computer-readable medium and configured to execute instructions to perform the functions described herein. It should be appreciated that at least a portion of one or more of the gait data collector 302, the gait data analyzer 304, the toe walking determiner 306, and/or the mobile application interface 308 may be located in the heel portion 102 and/or the forefoot portion 104.

The gait data collector 302, which may be embodied as any type of firmware, hardware, software, circuitry, or combination thereof, is configured to collect information from the sensors 410. To do so, the gait data collector 302 is configured to collect a measured force (e.g., via an electrical charge) from one or more of the load cells 412 at a given time. Additionally, the gait data collector 302 may be further configured to collect movement data of the gait monitoring device 100 relative to the support surface (e.g., the ground). The gait data collector 302 may be configured to poll (i.e., pulled by the gait data collector 302) or receive measurements (i.e., pushed from the sensors 410) at a regular interval and/or subsequent to a detected event, depending on the type of data and/or the particular embodiment. In some embodiments, the gait data collector 302 is configured to store the gait data in a database (not shown) that is usable to access the data post-storage to perform at least a portion of the functions described herein.

The gait data analyzer 304, which may be embodied as any type of firmware, hardware, software, circuitry, or combination thereof, is configured to analyze the data collected by the gait monitoring device 100 (e.g., via the gait data collector 302) to determine which phase of the gait cycle the wearer is presently in. Accordingly, the gait data analyzer 304 is configured to determine whether the wearer is presently moving on foot in a bipedal locomotion, as opposed to being at rest (e.g., sitting, lying down, etc.). To do so, the gait data analyzer 304 is configured to compare present gait data against one or more threshold values. Such movement threshold values may include any values usable to determine whether the wearer is presently moving on foot in a bipedal locomotion, such as a minimum acceleration threshold, an orientation threshold, a magnetic flux threshold, etc.

To determine which phase of the gait cycle the wearer is presently in, gait data analyzer 304 is configured to analyze the data collected from the load cell sensors 412, as well as the IMU sensors(s) 414, as may be available/necessary. It should be appreciated that the gait data analyzer 304 may be configured to use any technology for gait analysis (e.g., to determine a present phase/period of the gait cycle) known to those of skill in the art. To do so, the gait data analyzer 304 is configured to analyze the gait data collected by the gait monitoring device 100 and compare the received data against one or more load threshold values, such as a minimum detected weight, a minimum detected force, a minimum electrical charge, etc.

The toe walking determiner 306, which may be embodied as any type of firmware, hardware, software, circuitry, or combination thereof, is configured to determine whether a wearer is presently toe walking. Of course, it should be appreciated that the toe walking determiner 306 is configured to do so only upon a determination that the wearer is presently walking/running and the wearer is presently in the initial contact period of the stance phase of the gait cycle (e.g., as may be determined a result of the analysis performed by the gait data analyzer 304). In other words, the toe walking determiner 306 is configured to determine whether a heel strike is detected during the initial contact period. To do so, the toe walking determiner 306 is configured to compare data collected by the gait data collector 302 against one or more corresponding threshold values, such as the minimum detected weight, the minimum detected force, the minimum electrical charge, etc.

The toe walking determiner 306 is additionally configured to initiate the vibration circuitry 408 to notify the wearer in the event of a detected toe walking event. Accordingly, the toe walking determiner 306 is configured to communicate with the vibration circuitry 408. The toe walking determiner 306 is further configured to communicate the detection of the toe walking event to a corresponding toe walking tracker application 312 (e.g., via the mobile application interface 308). It should be appreciated that, in some embodiments, the toe walking determiner 306 may be configured to transmit such a toe walking event communication to the toe walking tracker application 312 either upon detection or during a batch upload (e.g., at a particular time of day, upon detection of a communication channel with the toe walking tracker application 312, upon receiving a request from the toe walking tracker application 312, etc.).

The mobile application interface 308, which may be embodied as any type of firmware, hardware, software, circuitry, or combination thereof, is configured to interface with a software application associated (i.e., linked, paired, etc.) with the gait monitoring device 100, such as the toe walking tracker application 312 described below. In other words, the mobile application interface 308 is configured to transmit messages to and receive messages from the toe walking tracker application 312. Accordingly, it should be appreciated that the gait monitoring device 100 is configured to instantiate a communication channel and wirelessly communicate said communication transmissions with the mobile computing device 310 on which the toe walking tracker application 312 is installed. Additionally, in some embodiments, the mobile application interface 308 may be configured to store one or more credentials (e.g., a key, a username, a password, etc.) received from a user via the toe walking tracker application 312 in a secure database such that the credential(s) may be used to secure the communication channel between the gait monitoring device 100 and the mobile computing device 310. In such embodiments, the credentials may be received and/or the communication channel may be instantiated via an out-of-band exchange of information.

The mobile computing device 310 may be embodied as any type of portable computing device capable of performing the functions described herein. Specifically, the mobile computing device 310 may be embodied as any type of portable computing device that uses mobile-specific hardware and software components for operating and executing on a mobile architecture. Illustrative examples of such a mobile computing device 310 include, but are not limited to, smartphones, wearables (e.g., smartwatches, smart glasses, etc.), tablets, laptops, etc. Accordingly, the mobile computing device 310 may include any type of firmware, hardware, software, circuitry, or combination thereof capable of performing the functions described herein.

Referring now to FIG. 5, the illustrative mobile computing device 310 includes a CPU 500, an I/O controller 502, a memory 504, network communication circuitry 506, one or more I/O peripherals 508, a data storage device 512, and various sensors 514. It should be appreciated that alternative embodiments may include additional, fewer, and/or alternative components to those of the illustrative mobile computing device 310, such as a graphics processing unit (GPU). It should be further appreciated that the mobile computing device 310 may contain like components to that of the illustrative gait monitoring device 100 of FIG. 4. Accordingly, such like components are not described herein to preserve clarity of the description.

The one or more I/O peripherals 508 may be embodied as any auxiliary device configured to connect to and communicate with the mobile computing device 310. For example, the I/O peripherals 508 may include, but are not limited to, a mouse, a keyboard, a monitor, a touchscreen display, a printer, a scanner, a microphone, a speaker, etc. Accordingly, it should be appreciated that some I/O peripherals 508 are capable of one function (i.e., input or output), or both functions (i.e., input and output). The illustrative I/O peripherals 508 includes a display 510, which may be embodied as a touchscreen display capable of receiving user input via touch (e.g., one or more fingers, a stylus, etc.) and outputting user interfacing elements (e.g., via a graphical user interface (GUI)).

The data storage device 512 may be embodied as any type of computer hardware capable of the non-volatile storage of data (e.g., semiconductor storage media, magnetic storage media, optical storage media, etc.). Such data storage devices 512 are commonly referred to as auxiliary or secondary storage, and are typically used to store a large amount of data relative to the main memory 504.

Referring back to FIG. 3, the illustrative mobile computing device 310 includes an toe walking tracker application 312 usable by the wearer and/or another monitoring party to track toe walking events for a wearer. The toe walking tracker application 312 may be embodied as any type of mobile-based software application, such as a thick client or a thin client (e.g., cloud application, network application, software-as-a-service (SaaS) application, etc.), that is configured to wirelessly communicate with the gait monitoring device 100. In some embodiments, the toe walking tracker application 312 is configured to store the gait data in a database (not shown) that is usable to access the data post-storage to perform at least a portion of the functions described herein.

The illustrative toe walking tracker application 312 includes a user interface 314, a gait data aggregator 316, a gait data analyzer 318, and a gait monitoring device interface 320, each of which may be embodied as any type of firmware, hardware, software, circuitry, or combination thereof that is configured to perform the functions described herein. In some embodiments, the user interface 314, the gait data aggregator 316, the gait data analyzer 318, and/or the gait monitoring device interface 320 may include one or more computer-readable medium (e.g., the main memory 504, the data storage device 512, and/or any other media storage device) having instructions stored thereon and one or more processors (e.g., the CPU 500) coupled with the one or more computer-readable medium and configured to execute instructions to perform the functions described herein. It should be appreciated that, in some embodiments (e.g., low storage/compute capacity gait monitoring devices 100), at least a portion of one or more of the functions described herein for the gait data analyzer 304 and/or the toe walking determiner 306 may be performed by the toe walking tracker application 312.

The mobile application interface 314, which may be embodied as any type of firmware, hardware, software, circuitry, or combination thereof, is configured to serve as an interface between the wearer (i.e., the owner/operator of the mobile computing device 310) and the gait monitoring device 100. In an illustrative embodiment, the user interface 314 is configured to provide one or more graphical user interfaces (GUIs) consisting of one or more GUI elements which are usable to output information to a user and receive input therefrom. Accordingly, the mobile application interface 314 may be used to facilitate the creation of a tracking account, login to the toe walking tracker application 312, establish a connection with the gait monitoring device 100, configure one or more settings of the gait monitoring device 100, calibrate one or more sensors of the gait monitoring device 100, review gait cycle data, review toe walking event data, etc.

The gait data aggregator 316, which may be embodied as any type of firmware, hardware, software, circuitry, or combination thereof, is configured to search, gather, and present the gait data and/or toe walking event data in a report-based, summarized format for user consumption. Accordingly, the summarized format may be visually presented to a user of the toe walking tracker application 312 in a more digestible format.

The gait data analyzer 318, which may be embodied as any type of firmware, hardware, software, circuitry, or combination thereof, is configured to perform functions similar to the gait data analyzer 304 of the gait monitoring device 100. Such functions may be performed in addition to the similar functions of the gait data analyzer 304 of the gait monitoring device 100 as described previously, or in replacement thereof. As described previously, some embodiments of the gait monitoring device 100 may not include the IMU sensor(s) 414.

Accordingly, in such embodiments, the gait data analyzer 318 may be the source of the analysis results used by the toe walking determiner 306. In other words, the gait data analyzer 318 may perform the analysis computations and return the results to the gait monitoring device 100 to determine whether a toe walking event was detected. It should be appreciated, however, that in some embodiments the toe walking event determination may additionally be performed by the gait data analyzer 318 rather than the toe walking determiner 306. In such embodiments, the gait data analyzer 318 is further configured to provide a toe walking event indication (e.g., via the gait monitoring device interface 320 to the mobile application interface 308) to trigger the vibration circuitry 408.

The gait monitoring device interface 320, which may be embodied as any type of firmware, hardware, software, circuitry, or combination thereof, is configured to interface with the gait monitoring device 100 (e.g., via the mobile application interface 308) to which the toe walking tracker application 312 has been associated (i.e., linked, paired, etc.). In other words, the gait monitoring device interface 320 is configured to transmit messages to and receive messages from the mobile application interface 308. Accordingly, it should be appreciated that mobile computing device 310 is configured to instantiate a communication channel and wirelessly communicate said communication transmissions with the gait monitoring device 100 on which the mobile application interface 308 resides.

Additionally, in some embodiments, the gait monitoring device interface 320 may be configured to store one or more credentials (e.g., a key, a username, a password, etc.) received from a user (e.g., via the user interface 314) in a secure database such that the credential(s) may be used to secure the communication channel between the gait monitoring device 100 and the mobile computing device 310. As described previously, the credentials may be received and/or the communication channel may be instantiated via an out-of-band exchange of information.

It should be appreciated that, in some embodiments, the gait data measured by the gait monitoring device 100 and transmitted to the toe walking tracker application 312 may be further transmitted to a remote server (not shown) for additional tracking and/or further aggregation and analysis that may be useful to determine one or more of the toe walking determination parameters. In an illustrative example, the gait data may be stored in a remote server accessible via a software application executable on a remote computing device (not shown) communicatively coupled to the remote server, such as by a medical professional.

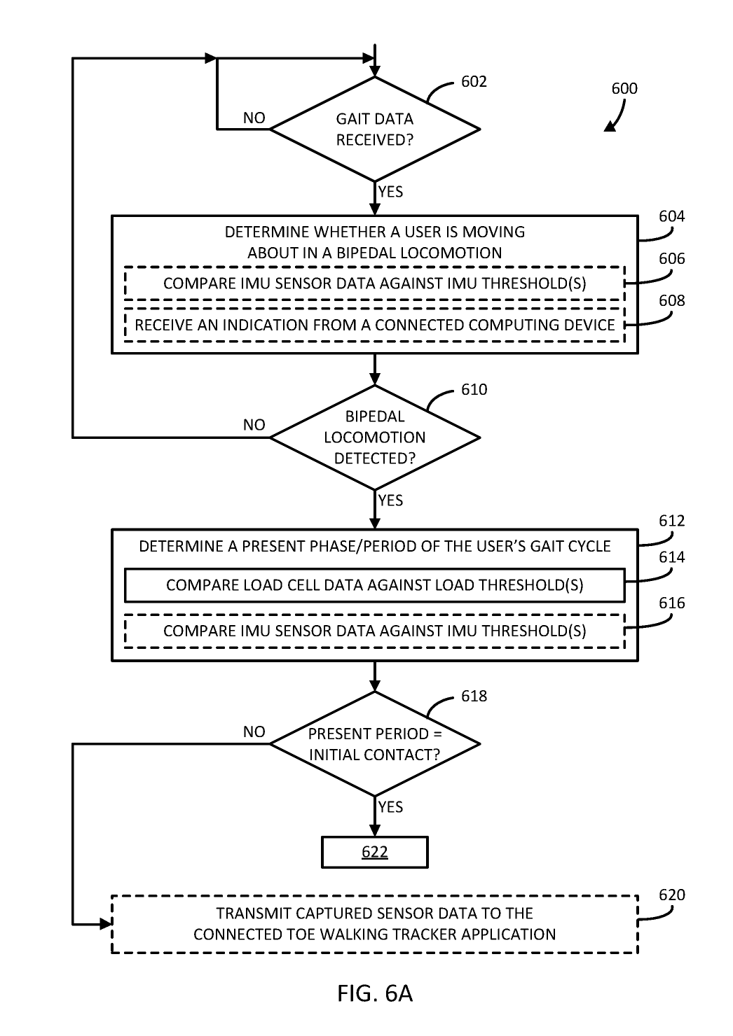

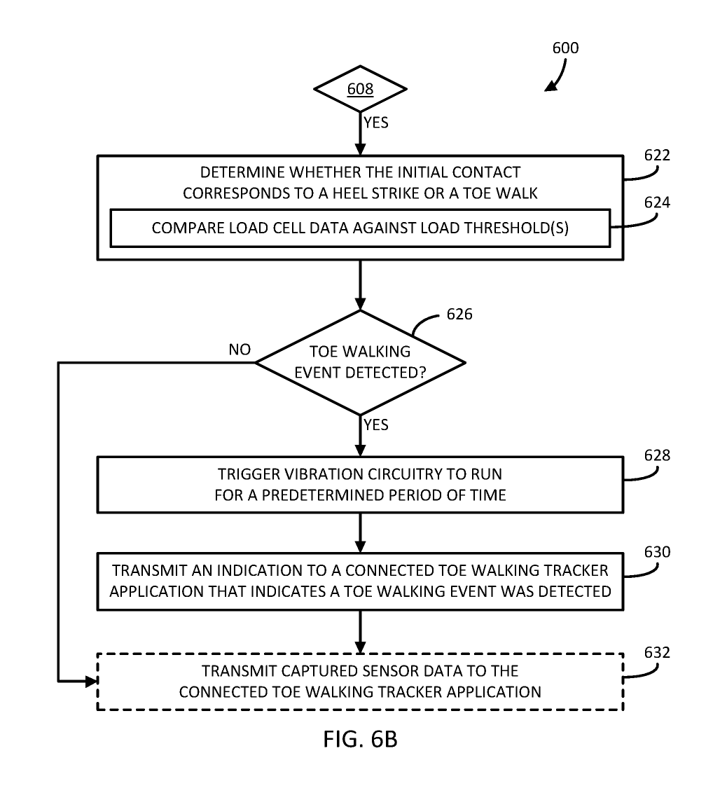

Referring now to FIGS. 6A and 6B, an illustrative method 600 is provided for indicating detection of an toe walking that may be executed by the gait monitoring device 100. It should be appreciated that, in some embodiments, one or more of the sensors may have been calibrated and/or a communication channel may have been established prior to the method 600 being invoked. The method 600 begins in block 602, in which the gait monitoring device 100 determines whether any gait data was received by the sensors 410 (e.g., via the gait data collector 302).

If so, the method 600 advances to block 604 in which the gait monitoring device 100 determines whether a user (i.e., a user of the gait monitoring device 100) is presently moving about on foot in a bipedal locomotion (i.e., walking, jogging, running, etc.) or not (e.g., via the gait data analyzer). To do so, in some embodiments, in block 606, the gait monitoring device 100 compares data collected by the IMU sensor(s) 414 against one or more corresponding threshold values. As described previously, such movement threshold values may include a minimum acceleration threshold, an orientation threshold, a magnetic flux threshold, etc. Alternatively, in other embodiments, in block 608, the gait monitoring device 100 may receive an indication from a connected computing device (e.g., the mobile computing device 310 of FIG. 3) that informs the gait monitoring device 100 that the gait monitoring device 100 is presently moving about on foot in a bipedal locomotion.

Accordingly, the gait monitoring device 100 can determine, in block 610, whether any detected movement associated with the gait monitoring device 100 is a bipedal locomotion or not. If the gait monitoring device 100 determines the gait monitoring device 100 is presently in bipedal locomotion, the method 600 advances to block 612; otherwise, the method 600 returns to block 602. In block 612, the gait monitoring device 100 determines a present phase and period of the wearer's gait cycle. To do so, in block 614, the gait monitoring device 100 is configured to compare collected load cell sensor 412 data against one or more load threshold values (e.g., a minimum detected weight, a minimum detected force, a minimum electrical charge, etc.). In some embodiments, in block 616, the gait monitoring device 100 is additionally configured to compare the IMU sensor 414 data against one or more of the movement threshold values.

Accordingly, the gait monitoring device 100 can determine, in block 618, whether the present period is an initial contact period of the stance phase. If not, in some embodiments, the method 600 branches to block 620 in which the gait monitoring device 100 transmits (e.g., via the mobile application interface 308) at least a portion of the gait data received in block 602 to a corresponding toe walking tracker application 312. Otherwise, if the gait monitoring device 100 determines the present phase corresponds to the initial contact period of the stance phase, the method 600 advances to block 622, as shown in FIG. 6B.

In block 622, the gait monitoring device 100 determines whether the initial contact corresponds to a heel strike or a toe walk event). To do so, the gait monitoring device 100 compares load cell sensor 412 data against one or more load threshold values to determines whether a user's heel is in contact with the sole of the shoe at the point of initial contact of the stance phase (i.e., a normal heel strike), as opposed to the user's heel being elevated and the user's forefoot being in contact with the forefoot portion of the shoe at the point of initial contact of the stance phase (i.e., a toe walking event). Accordingly, if the gait monitoring device 100 detects a toe walking event in block 626, the method 600 advances to block 628; otherwise, the method 600 jumps to block 632, which is described below.

In block 628, the gait monitoring device 100 triggers (i.e., enables) the vibration circuitry 408 to run for a predetermined period of time (e.g., a sufficient period of time such that the user can detect the vibration). In block 630, the gait monitoring device 100 transmits an indication (e.g., a message, a packet, etc.) to a connected toe walking tracker application 312 that indicates a toe walking event was detected such that the toe walking tracker application 312 can log the toe walking event and report the toe walking event to the user. In some embodiments, in block 632, the gait monitoring device 100 transmits (e.g., via the mobile application interface 308) at least a portion of the gait data received in block 602 to the connected toe walking tracker application 312.

While the present disclosure has been illustrated and described in detail in the drawings and foregoing description, the same is to be considered as illustrative and not restrictive in character, it being understood that only certain embodiments have been shown and described, and that all changes and modifications that come within the spirit of the present disclosure are desired to be protected.

What is claimed is:

1. A method for indicating detection of toe walking, the method comprising:
receiving, by a gait monitoring device, gait data from one or more sensors of the gait monitoring device;
detecting, by the gait monitoring device, a toe walking event as a function of the received gait data; and
enabling, by the gait monitoring device and subsequent to detecting the toe walking event, vibration circuitry of the gait monitoring device for a predetermined period of time.

2. The method of claim 1, further comprising determining, by the gait monitoring device, whether the gait monitoring device is being moved in a bipedal locomotion, wherein detecting the toe walking event is subsequent to having determined the gait monitoring device is being moved in the bipedal locomotion.

3. The method of claim 2, wherein determining whether the gait monitoring device is being moved in the bipedal locomotion comprises comparing at least a portion of the gait data received from an inertial measurement unit sensor of the gait monitoring device and one or more movement threshold values.

4. The method of claim 3, wherein the one or more movement threshold values include at least one of a minimum acceleration threshold, an orientation threshold, and a magnetic flux threshold.

5. The method of claim 1, further comprising determining, by the gait monitoring device, a present phase and a present period of a gait cycle of a user of the gait monitoring device, wherein detecting the toe walking event is subsequent to having determined the present phase of the gait cycle corresponds to a stance phase and the present period of the gait cycle corresponds to an initial contact period.

6. The method of claim 5, wherein determining the present phase and the present period of the gait cycle of the user comprises comparing at least a portion of the gait data received from a load cell sensor of the gait monitoring device and one or more load threshold values.

7. The method of claim 6, wherein the one or more load threshold values include at least one of a minimum detected weight, a minimum detected force, a minimum electrical charge.

8. The method of claim 1, further comprising transmitting, by the gait monitoring device, an indication to a toe walking tracker application presently executing on a mobile computing device that is wirelessly coupled to the gait monitoring device.

9. The method of claim 1, wherein detecting the toe walking event as a function of the received gait data comprises comparing at least a portion of the gait data received from a load cell sensor of the gait monitoring device and one or more load threshold values.

10. A gait monitoring device for indicating detection of toe walking, the gait monitoring device comprising:
one or more sensors usable to collect gait data relative to the gait monitoring device;
vibration circuitry that includes a vibrating motor usable to provide haptic feedback in the form of a vibration;
one or more computer-readable medium comprising instructions;
one or more processors coupled with the one or more computer-readable medium and configured to execute the instructions to:
analyze the gait data collected from the one or more sensors;
detect a toe walking event as a function of the analysis of the collected gait data; and
enable, subsequent to having detected the toe walking event, the vibrating motor for a predetermined period of time.

11. The gait monitoring device of claim 10, wherein the one or more processors are further configured to execute the instructions to determine whether the gait monitoring device is being moved in a bipedal locomotion, wherein to detect the toe walking event is subsequent to having determined the gait monitoring device is being moved in the bipedal locomotion.

13. The gait monitoring device of claim 12, wherein to determine whether the gait monitoring device is being moved in the bipedal locomotion comprises to compare at least a portion of the gait data received from an inertial measurement unit sensor of the gait monitoring device and one or more movement threshold values.

14. The gait monitoring device of claim 13, wherein the one or more movement threshold values include at least one of a minimum acceleration threshold, an orientation threshold, and a magnetic flux threshold.

15. The gait monitoring device of claim 11, wherein the one or more processors are further configured to execute the instructions to determine a present phase and a present period of a gait cycle of a user of the gait monitoring device, wherein to detect the toe walking event is subsequent to having determined the present phase of the gait cycle corresponds to a stance phase and the present period of the gait cycle corresponds to an initial contact period.

16. The gait monitoring device of claim 15, wherein the one or more sensors includes one or more load cell sensors, and wherein to determine the present phase and the present period of the gait cycle of the user comprises comparing at least a portion of the gait data received from at least one of the load cell sensors and one or more load threshold values.

17. The gait monitoring device of claim 16, wherein the one or more load threshold values include at least one of a minimum detected weight, a minimum detected force, a minimum electrical charge.

18. The gait monitoring device of claim 11, wherein the one or more processors are further configured to transmit an indication to a toe walking tracker application presently being executed on a mobile computing device that is wirelessly coupled to the gait monitoring device.

19. The gait monitoring device of claim 11, wherein the one or more sensors includes one or more load cell sensors, and wherein to detect the toe walking event as a function of the received gait data comprises comparing at least a portion of the gait data received from at least one of the load cell sensors and one or more load threshold values.
